(19) United States
(12) Patent Application Publication
(10) Pub. No.: US 2025/0046448 A1
(43) Pub. Date: Feb. 6, 2025

(54) Remote Point of Care Patient Care System and Methods

(71) Applicant: Diagenta Inc., San Diego, CA (US)

(72) Inventors: Gelena Lifchitz, San Marcos, CA (US); Teresa Sunder Prince, La Jolla, CA (US)

(21) Appl. No.: 18/363,838

(22) Filed: Aug. 2, 2023

(51) Int. Cl. G16H 50/20; G16H 10/60

(52) U.S. Cl. CPC G16H 50/20; G16H 10/60

(57) ABSTRACT

The Remote Point of Care Patient Care System and Methods is a remotely user operated point of care patient care system for getting patient input, conducting patient tests, and reporting test results. This system allows users to have an easy and convenient way to decrease diagnostic error, provide faster diagnosis, treatment, and prophylaxis support decisions, and set up onsite diagnostic support systems. This system can make tasks easier and more convenient because of its earlier, deeper, and more complete integration of both new and old information into the remote patient care provider experience.

### TECHNICAL FIELD OF THE INVENTION

This invention relates generally to a point of care patient care system for getting patient input, conducting patient tests, and reporting test results.

This invention relates more specifically to a remotely user operated point of care patient care system for getting patient input, conducting patient tests, and reporting test results.

This invention relates generally to a point of care patient care system for getting patient input, conducting patient tests, and reporting test results that enables communication with a patient care provider for diagnosis and treatment of a patient.

This invention relates generally to a remotely user operated point of care patient care system for getting patient input, conducting patient tests, and reporting test results that enables communication with a patient care provider for diagnosis and treatment of a patient.

### BACKGROUND

There are systems, apparatuses, and devices available today that offer point of care patient care, patient condition, and patient history reporting. There are those that enable some physical examination, some bodily fluid testing. There are systems, apparatuses, and devices that support diagnosis, treatment, and prophylaxis to include computers/laptops, mobile devices, webcams, microphones, headsets, telemedicine software/applications, electronic health record (EHR) systems, diagnostic devices (stethoscopes, thermometers, blood pressure monitors, pulse oximeters, glucometers), imaging equipment, remote monitoring devices, and secure communication tools to connect systems, apparatuses, and devices.

Currently available systems, apparatuses, and devices suffer from diagnostic errors and slow time to diagnosis, treatment, and prophylaxis support decisions. There is also a lack of onsite diagnostic support systems.

Lacking in the prior art is a unified point of care remote patient care system that allows users an easy and convenient way to decrease diagnostic error, seek and obtain faster diagnosis, treatment, and prophylaxis support decisions than are available today, and get onsite diagnostic support systems available to help both patients and care providers.

The present invention is not just a new business process, it is a joint experience between a patient and a care provider that enables a unified point of care remote patient care system. This system allows users to have an easy and convenient way to decrease diagnostic error, provide faster diagnosis, treatment, and prophylaxis support decisions, and set up onsite diagnostic support systems. This system can make tasks easier and more convenient because of its earlier, deeper, and more complete integration of both new and old information into the remote patient care provider experience. The present invention offers more complete historical content, enhanced range of test devices and tests available, deeper by more complete and integrated new patient vitals and test results.

In light of the foregoing prior art, there is a need for a unified point of care remote patient care system that allows users an easy and convenient way to decrease diagnostic error, generate faster more accurate diagnosis, treatment, and prophylaxis support decisions, and create onsite diagnostic support systems.

### BRIEF SUMMARY OF THE INVENTION

The present invention is a mobile diagnostic support system is also known as a point of care (POC) device for provisioning of medical care based on patient history, patient condition reports, physical examination, chemical examination, and tests of conditions of bodily fluids and gases.

According to a first aspect of the invention, there is a mobile diagnostic support system for enhanced integration and involvement between a patient user and a care provider for provisioning medical care to a patient user comprising a plurality of user symptom reports (USR), one of said plurality of USR comprising a first user health condition report, a plurality of exam devices (ED), one of said plurality of ED comprising a vital sign monitor (VSM) having a first user exam result (UER), a plurality of lab tests (LT), one of said plurality of LT comprising a bodily fluid test (BFT) having a first user test result (UTR), a database comprising analysis data, said analysis data comprising a plurality of USR data, one of said plurality of USR data comprising a symptom report, a plurality of UER data, one of said plurality of UER data comprising a VSM exam result, a plurality of UTR data, one of said plurality of UTR data comprising a BFT test result, a plurality of diagnosis data (DD) one of said plurality of DD comprising a plurality of ED diagnosis interpretations (ED-DI) one of said plurality of ED-DI comprising a BPM (blood pressure monitor) diagnosis interpretation (BPM-DI) dataset, a second of said plurality of ED-DI comprising an LT diagnosis interpretation (LT-DI) dataset, a plurality of treatment data (TD), one of said plurality of TD comprising a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and a plurality of prophylaxis data (PD), one of said plurality of PD comprising a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset, an algorithm, a user device, a processor, and a care provider device, wherein optionally said user inputs at least one user health condition report, wherein said user connects at least one of said plurality of ED and/or said plurality of LT, wherein said processor uses said algorithm via encryption secured electronic transmission to connect to said plurality of ED and/or said plurality of LT, identify each of said plurality of ED and/or said plurality of LT, read a data from each of said plurality of ED and/or said plurality of LT, reformat/reorganize said data into a standard/consolidated formatted data-stream, and transmit said standard/consolidated data-stream into and/or attach to a patient record system, wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion, wherein said care provider device is used by a care provider to offer onsite diagnostic support, and wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

According to a second aspect of the invention, there is a mobile diagnostic support system wherein said database further comprises a plurality of medical billing data (MBD), one of said plurality of MBD comprising a medical device billing data (MDBD), and wherein said processor uses said algorithm via encryption secured electronic transmission to display a billing suggestion.

According to a third aspect of the invention, there is a mobile diagnostic support system further comprising a chatbot to review, assess, comment on, and/or adjust any of said plurality of USR, said plurality of UER, said plurality of UTR, said diagnosis suggestion, said treatment suggestion, and said prophylaxis suggestion.

According to a fourth aspect of the invention, there is a mobile diagnostic support system further comprising a chatbot to review, assess, comment on, and/or adjust said plurality of MBD and/or said billing suggestion.

According to a fifth aspect of the invention, there is a mobile diagnostic kit including the mobile diagnostic support system comprising: a backpack; a wheeled case; a stethoscope; a video camera; and a vital signs monitor comprising measurements for any combination of temperature, blood pressure, oxygen saturation, and/or heart rate.

According to a sixth aspect of the invention, there is a mobile diagnostic kit further comprising any combination of: a blood chemistry analysis apparatus; and/or a hematology analysis apparatus.

According to a seventh aspect of the invention, there is a mobile diagnostic kit further comprising: a urine analysis apparatus.

According to an eighth aspect of the invention, there is a mobile diagnostic kit further comprising any combination of an electrocardiography analysis apparatus; a digital spirometer; and an ultrasound analysis apparatus.

According to a ninth aspect of the invention, there is a mobile diagnostic kit further comprising any combination of: a blood clot analysis apparatus; an antigen presence analysis apparatus; a blood lipid analysis apparatus; a glycated hemoglobin analysis apparatus; and/or a rapid molecular test apparatus.

According to a tenth aspect of the invention, there is a mobile diagnostic kit further comprising a recurring subscription fee.

According to an eleventh aspect of the invention, there is a method for enhanced integration and involvement between a patient user and a care provider during a provisioning of medical care to a patient user comprising a plurality of user symptom reports (USR), one of said plurality of USR comprising a first user health condition report, a plurality of exam devices (ED), one of said plurality of ED comprising a vital sign monitor (VSM) having a first user exam result (UER), a plurality of lab tests (LT), one of said plurality of LT comprising a bodily fluid test (BFT) having a first user test result (UTR), a database comprising analysis data, said analysis data comprising a plurality of USR data, one of said plurality of USR data comprising a symptom report, a plurality of UER data, one of said plurality of UER data comprising a VSM exam result, a plurality of UTR data, one of said plurality of UTR data comprising a BFT test result, a plurality of diagnosis data (DD) one of said plurality of DD comprising a plurality of ED diagnosis interpretations (ED-DI) one of said plurality of ED-DI comprising a BPM diagnosis interpretation (BPM-DI) dataset, a second of said plurality of ED-DI comprising an LT diagnosis interpretation (LT-DI) dataset, a plurality of treatment data (TD), one of said plurality of TD comprising a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and a plurality of prophylaxis data (PD), one of said plurality of PD comprising a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset, an algorithm, a user device, a processor, and a care provider device, wherein optionally said user inputs at least one user health condition report, wherein said user connects at least one of said plurality of ED and/or said plurality of LT, wherein said processor uses said algorithm via encryption secured electronic transmission to connect to said plurality of ED and/or said plurality of LT, identify each of said plurality of ED and/or said plurality of LT, read a data from each of said plurality of ED and/or said plurality of LT, reformat/reorganize said data into a standard/consolidated formatted data-stream, and transmit said standard/consolidated data-stream into and/or attach to a patient record system, wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion, wherein said care provider device is used by a care provider to offer onsite diagnostic support, and wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

According to a twelfth aspect of the invention, there is a method wherein said database further comprises a plurality of medical billing data (MBD), one of said plurality of MBD comprising a medical device billing data (MDBD), and wherein said processor uses said algorithm via encryption secured electronic transmission to display a billing suggestion.

According to a thirteenth aspect of the invention, there is a method further comprising a chatbot to review, assess, comment on, and/or adjust any of said plurality of USR, said plurality of UER, said plurality of UTR, said diagnosis suggestion, said treatment suggestion, and said prophylaxis suggestion.

According to a fourteenth aspect of the invention, there is a method further comprising a chatbot to review, assess, comment on, and/or adjust said plurality of MBD and/or said billing suggestion.

According to a fifteenth aspect of the invention, there is a mobile diagnostic kit for implementing a method for enhanced integration and involvement between a patient user and a care provider during a provisioning of medical care to a patient user comprising: a backpack; a wheeled case; a stethoscope; a video camera; and a vital signs monitor comprising measurements for any combination of temperature, blood pressure, oxygen satiation, and heart rate.

According to a sixteenth aspect of the invention, there is a mobile diagnostic kit further comprising any combination of: a blood chemistry analysis apparatus; and/or a hematology analysis apparatus.

According to a seventeenth aspect of the invention, there is a mobile diagnostic kit further comprising: a urine analysis apparatus.

According to a eighteenth aspect of the invention, there is a mobile diagnostic kit further comprising any combination of an electrocardiography analysis apparatus; a digital spirometer; and an ultrasound analysis apparatus.

According to a nineteenth aspect of the invention, there is a mobile diagnostic kit further comprising any combination of: a blood clot analysis apparatus; an antigen presence analysis apparatus; a blood lipid analysis apparatus; a glycated hemoglobin analysis apparatus; and/or a rapid molecular test apparatus.

According to a twentieth aspect of the invention, there is a mobile diagnostic kit further comprising a recurring subscription fee.

An advantage of the remote patient care system is that it offers a unified POC remote patient care system that allows users an easy and convenient way to decrease diagnostic error, offer faster (shorter) time to diagnosis, treatment, and prophylaxis support decisions, and create onsite diagnostic support system.

The invention will now be described, by way of example only, with reference to the accompanying drawings in which:

### BRIEF DESCRIPTION OF THE DRAWINGS

FIG. 1 is a diagram of the remote POC patient care system and methods according to the invention; and

FIG. 2 is a flowchart diagram of the remote POC patient care system and methods according to the invention.

### DETAILED DESCRIPTION

The detailed embodiments of the present invention are disclosed herein. The disclosed embodiments are merely exemplary of the invention, which may be embodied in various forms. The details disclosed herein are not to be interpreted as limiting, but merely as the basis for the claims and as a basis for teaching one skilled in the art how to make and use the invention.

References in the specification to “one embodiment,” “an embodiment,” “an example embodiment,” etcetera, indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to effect such feature, structure, or characteristic in connection with other embodiments whether or not explicitly described.

Furthermore, it should be understood that spatial descriptions (e.g., “above,” “below,” “up,” “left,” “right,” “down,” “top,” “bottom,” “vertical,” “horizontal,” etc.) used herein are for purposes of illustration only, and that practical implementations of the structures described herein can be spatially arranged in any orientation or manner.

Throughout this specification, the word “comprise”, or variations thereof such as “comprises” or “comprising”, will be understood to imply the inclusion of a stated element, integer or step, or group of elements integers or steps, but not the exclusion of any other element, integer or step, or group of elements, integers or steps.

The present invention is a mobile diagnostic support system, also known as a POC device, usually in the form of a handheld computing device configured for provisioning of medical care based on patient history, patient condition reports, physical examination, chemical examination, and tests of conditions of bodily fluids and gases. A user, or patient, operated device is connected through an algorithm to electronic patient records, tests, reports, and connected services in use. The patient operated device is connected to a care provider for a POC remote patient care system that provides users with an easy and convenient way to decrease diagnostic error, seek and obtain faster diagnosis, treatment, and prophylaxis support decisions than are available today, and get onsite diagnostic support systems available to help both patients and care providers.

The patient operated device is configured to connect the analysis results and test results of at least, but not limited to: a blood chemistry analyzer; a hematology analyzer; a urine analyzer; a digital stethoscope; a video camera; a vital signs monitor comprising measurements for temperature, blood pressure, oxygen satiation, and heart rate; an electrocardiography analyzer; an ultrasound device; a blood clot analyzer; an antigen presence analyzer; a blood lipid analyzer; a glycated hemoglobin analyzer; and a rapid molecular test apparatus.

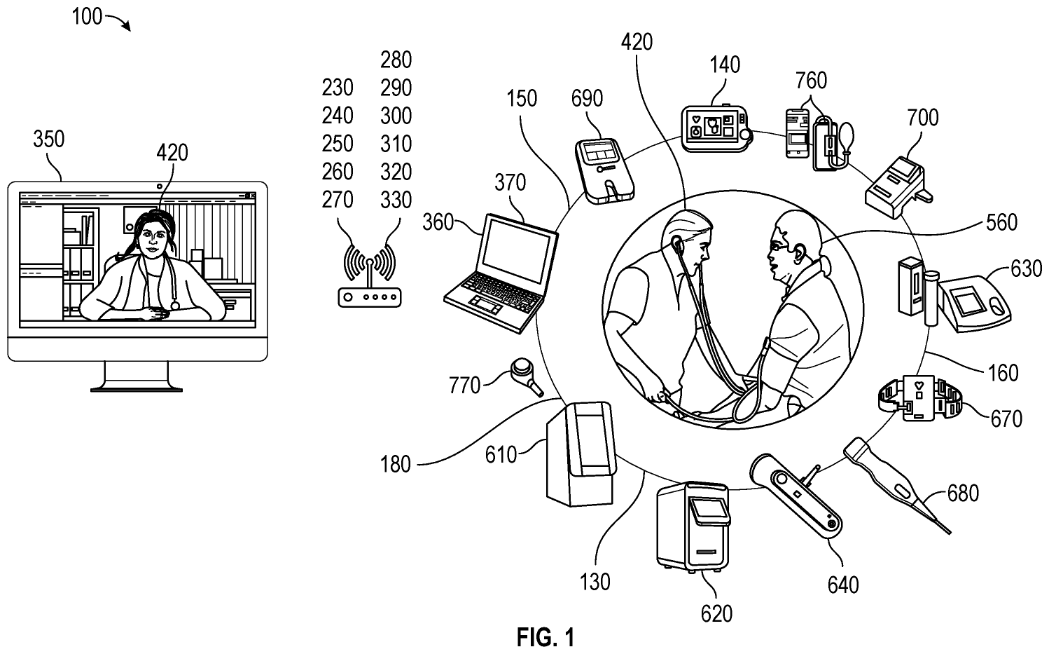

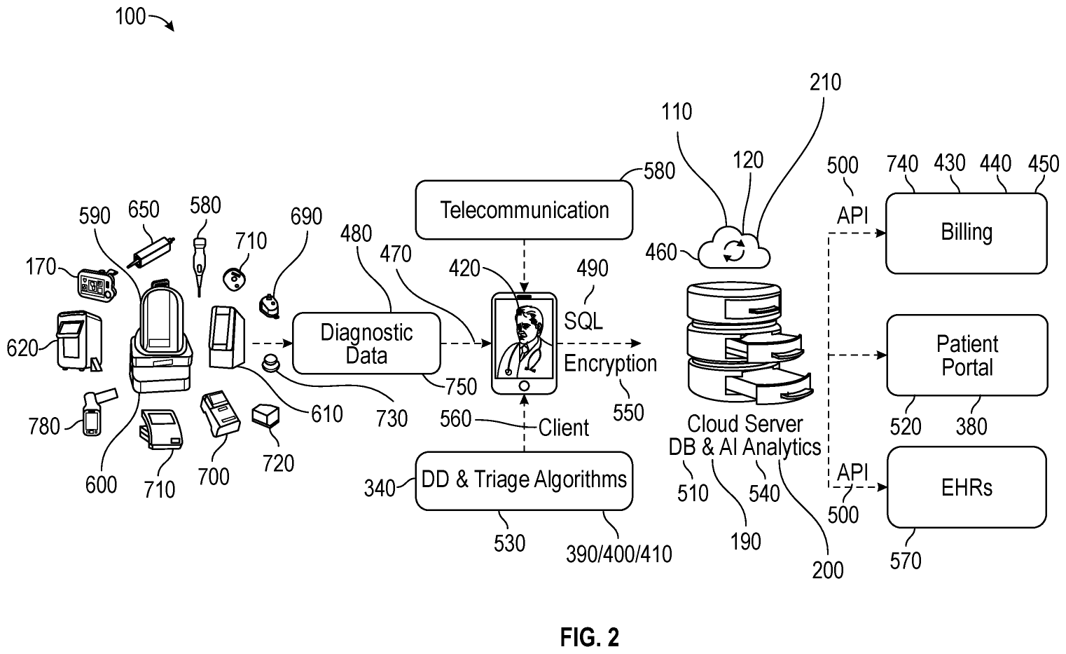

Referring to the Figures, there is shown in FIGS. 1 and 2 the following features of the invention claimed listed in numerical order.

Element 100 which is a mobile diagnostic support system.

Element 110 which is a USR (user symptom report).

Element 120 which is a user health condition report.

Element 130 which is an ED (exam device).

Element 140 which is a VSM (vital sign monitor).

Element 150 which is a UER (user exam result).

Element 160 which is a LT (lab test).

Element 170 which is a BFT (bodily fluid test).

Element 180 which is a UTR (user test result).

Element 190 which is a database.

Element 200 which is analysis data.

Element 210 which is a symptom report.

Element 230 which is an ED-DI (ED diagnosis interpretation) dataset.

Element 240 which is a BPM-DI (BPM diagnosis interpretation) dataset.

Element 250 which is an LT-DI (LT diagnosis interpretation) dataset.

Element 260 which is TD (treatment data).

Element 270 which is an ED-TI (ED treatment interpretation) dataset.

Element 280 which is a BPM-TI (BPM treatment interpretation) dataset.

Element 290 which is an LT-TI (LT treatment interpretation) dataset.

Element 300 which is PD (prophylaxis data).

Element 310 which is an ED-PI (ED prophylaxis interpretation) dataset.

Element 320 which is a BPM-PI (BPM prophylaxis interpretation) dataset.

Element 330 which is an LT-PI (LT prophylaxis interpretation) dataset.

Element 340 which is an algorithm.

Element 350 which is a user device.

Element 360 which is a processor.

Element 370 which is a care provider device.

Element 380 which is a patient record system.

Element 390 which is a diagnosis suggestion.

Element 400 which is a treatment suggestion.

Element 410 which is a prophylaxis suggestion.

Element 420 which is a care provider.

Element 430 which is MBD (medical billing data).

Element 440 which is MDBD (medical device billing data).

Element 450 which is a billing suggestion.

Element 460 which is a chatbot.

Element 470 which is a standard/consolidated data-stream.

Element 480 which is data.

Element 490 which is SQL (server query language).

Element 500 which is a API (automated programming interface).

Element 510 which is a Cloud Server DB (database).

Element 520 which is a patient portal.

Element 530 which is a triage algorithm.

Element 540 which is AI (artificial intelligence) analytics.

Element 550 which is an encryption.

Element 560 which is a client.

Element 570 which is an EHR (employee health record).

Element 580 which is telecommunication.

Element 590 which is a backpack.

Element 600 which is a wheeled case.

Element 610 which is a blood chemistry analyzer.

Element 620 which is a hematology analyzer.

Element 630 which is a urine analyzer.

Element 640 which is a digital stethoscope.

Element 650 which is a video camera.

Element 670 which is an electrocardiography analyzer.

Element 680 which is an ultrasound device.

Element 690 which is a blood clot analyzer.

Element 700 which is an antigen presence analyzer.

Element 710 which is a blood lipid analyzer.

Element 720 which is a glycated hemoglobin analyzer.

Element 730 which is a rapid molecular test apparatus.

Element 740 which is billing.

Element 750 which is diagnostic data.

Element 760 which is a blood pressure monitor.

Element 770 which is a liquid blood collection device.

Element 780 which is digital spirometer.

In an embodiment, the present invention is a POC device for provisioning medical care to a patient in form of a user/patient operated handheld device connected to electronic records and a care provider device to enable provisioning of patient care to said user by said care provider. This includes at least an embodiment comprised of a portable computing device with the proprietary software, also called the Diagenta system, Diagenta software, or Diagenta algorithm. The usually connected electronic records include but is not limited to: patient records integrated with POC devices and HIPAA (Health Insurance Portability and Accountability Act) compliant telecommunications. The Diagenta software enables a mobile diagnostic support system in the form of a POC device such as a smart phone, or tablet computing device.

The software of the present invention is operated in the usual client server arrangement and/or in the form of a web-enabled computer application. While any existing computing technology may be configured to enable the software of the present invention, preferred embodiments make use of smart phone and tablet devices as the usual form factor for the user or patient device and make use of tablet devices or laptop devices. The present invention offers quicker interaction time earlier in the process and testing to gain that leverage for offering something more than a mere abstract idea of POC solutions. This combined with the wide and large array of views, tests, and measurements available and the availability of onsite diagnostic support system from care provider to user patient makes the present invention superior.

The present invention is rapid diagnostics delivered by medical technicians with triage and treatment provided under remote oversight. The present invention is a “clinic without walls,” or a user portable POC device that can be used at a patient's home, on mobile care provisioning van, or even from inside the trunk of a nurse's or technician's car.

The present invention is configurable as a device and/or a method for in home self use, or mobile care provider use. The systems, apparatuses, and devices of the present invention offer decreased error in diagnosis, treatment, and prophylaxis due to the increased interaction and increased speed of application/use due to increased interaction and the wider and larger array of available vitals signs, tests, views, and reports. Optionally the systems, apparatuses, and devices of the present invention can also be configured as a user operated diagnostic chatbot AI, that can escalate to video care provider.

The present invention is configurable to provide while a patient user is video connected to a care provider to offer at least all of the following: decision-making support; smart triage tool; patient risk assessment; and workflow optimization. Each offered in all of the usual means and methods available today within the given the specific technology implementation for and given part, element, device, apparatus, or component under consideration. Implementing these systems is done with the presently available tools in the art. It is the enhanced interactions between patient users and care providers combined with the wide range and array of tests, measurements, and reports available that offer much more than the combination in itself. The depth and range of interaction is wide and deep between patient users and care providers, this increase in interaction and the combination of available tests, measures, and results offers much more for most of the common (at least all of the 85% most common) aliments that patients suffer from today are dealt with and addressed faster and more completely with the present invention than anything previously available.

The present invention is configurable to offer enhanced patient to care provider communication to include but not be limited to: on-line scheduling specific to house calls and virtual visits; AI driven diagnostic chatbot; secured access to health records; on-line appointment scheduling; billing and payment; and/or provider feedback and rating services.

The present invention is configurable as a device and/or a method for faster patient treatment decisions in the diagnosis-intervention-prevention care loop to include diagnosis, intervention, and prevention and treatment decision support in 15 minutes or less. This is including diagnostic chatbot AI, and in full in home exams list to address all common maladies.

The present invention is configurable as a device and/or a method for remote physical examination to include but not to limited to at least the following: digital auscultation, video-monitoring; vitals: temperature, oxygen saturation in blood, blood pressure, heart rate, and respiration rate; up to 12-lead ECG (electrocardiograph); digital spirometry; heart and lung ultrasound; abdominal and Pelvic ultrasound, bladder volume, deep vein thrombosis; and ultrasound.

The present invention is configurable as a device and/or a method to include within fifteen minute lab results or at least the following; Complete Blood Count with 5-diff; Comprehensive Metabolic Panel; Renal Panel, Liver Panel, Electrolytes; Diabetes Panel (glucose, A1C, ketones, cholesterol, lipids); Cardiac and Coagulation (Troponin, BNP, PT/INR); Urine analysis with rapid culture; Respiratory swabs (COVID, Flu, Strep, RSV); and COVID Panel (D-dimer, CRP, PT/INR, CBC, CMP).

In a preferred embodiment of the present invention, there is a DiaBag Mobile Connected Diagnostic System comprised of: a laptop personal computer with proprietary Diagenta software; patient records, integrated with POC devices and HIPAA-compliant tele-communicator; Piccolo Xpress Analyzer (blood chemistry); HemoScreen Portable Complete Blood Count Device (hematology); Accustrip Urine Analyzer (urine analyzer); Cue Health Rapid PCR (polymerase chain reaction) for Covid-19 detection-(a rapid molecular test apparatus); a Rapid Pathogen detector (an antigen presence analyzer): CardioChek Plus glucose, cholesterol and lipid spectrum analyzer (a blood lipid analyzer); HbA1C NOW+ Professional multi-test system (a glycated hemoglobin analyzer); Coag-Sense PT/INR PT2 Monitoring System (blood clot monitor); Nonagon N9+ Digital Stethoscope; Camera; Vitals monitor: Temp, BP, SpO2, HR; SHL-12 lead ECG with a belt/self-applied (an electrocardiography analyzer); Handheld ultrasound device Butterfly IQ (an ultrasound device); Durable backpack and wheeled suitcase for medical equipment; and a DiaLogic Monthly Subscription Fee.

The present invention is configurable as a device and/or a method customizable for specific house calls by car based operating care providers.

The present invention is configurable as a device and/or a method to use in connection with any available EHR and any current technology tele-health device, apparatus, or application.

The present invention is configurable as a device and/or a method to leverage, or use AI send chatbot systems.

A chatbot is a computer program or an AI system designed to simulate human conversation and interact with users through text or speech-based interfaces. It is typically used to provide automated responses and assist users in tasks such as answering questions, providing information, or completing specific actions.

Chatbots use natural language processing (NLP) techniques to understand and interpret user input and generate appropriate responses. They are trained on vast amounts of data and often employ machine learning algorithms to improve their performance over time.

Chatbots can be implemented in various forms, such as web-based chat interfaces, messaging apps, or voice assistants. They can be used in a wide range of applications, including customer service, virtual assistants, information retrieval, e-commerce, and more.

There are two primary types of chatbots:

Rule-based chatbots: These chatbots follow a predefined set of rules and patterns to respond to user input. They are programmed with specific responses for certain keywords or phrases and are limited to the rules they have been explicitly programmed with.

AI-powered chatbots: These chatbots use advanced machine learning algorithms and NLP techniques to understand and respond to user input. They can analyze the context, infer meaning, and generate more dynamic responses. AI-powered chatbots can learn from conversations, adapt their responses based on user interactions, and provide more personalized and conversational experiences.

Overall, chatbots aim to provide quick and efficient support, automate tasks, and enhance user experiences by simulating human-like conversations.

A chatbot for medical triage is a specialized type of chatbot designed to assist with assessing and determining the urgency and severity of medical conditions. Its primary purpose is to help individuals evaluate their symptoms, provide appropriate recommendations, and guide them to seek appropriate medical care.

Medical triage chatbots typically use a combination of predefined rules and advanced algorithms to analyze user-provided information about their symptoms and medical history. They may ask a series of questions to gather relevant details and use decision trees or algorithms to classify the symptoms based on their severity and urgency.

The chatbot evaluates the user's input against a database of medical knowledge and guidelines, which can include symptoms, conditions, treatments, and recommended actions. Based on this analysis, the chatbot provides personalized recommendations to the user, such as self-care advice, home remedies, suggestions to visit a doctor, or to seek emergency medical attention.

The advantages of using a medical triage chatbot include:

Accessibility: Chatbots can be accessed through various platforms like websites or messaging apps, allowing users to seek medical advice conveniently from their preferred devices.

Time-saving: Users can receive instant responses and guidance without the need for scheduling appointments or waiting for a healthcare professional's availability.

Scalability: Chatbots can handle multiple conversations simultaneously, ensuring that users can access the service without facing long wait times.

Risk assessment: By following established medical guidelines, chatbots can help identify potentially serious conditions that require urgent medical attention, ensuring timely intervention.

Pre-screening: Chatbots can help filter cases that require immediate attention, enabling healthcare providers to prioritize and allocate resources efficiently.

However, it's important to note that medical triage chatbots are not intended to replace medical professionals. They serve as a supportive tool to provide initial assessments and recommendations. If a user's symptoms are severe, rapidly worsening, or if there is uncertainty, it is always advisable to consult a qualified healthcare professional for a comprehensive evaluation and diagnosis.

A chatbot for medical treatment is a specialized type of chatbot designed to provide assistance and support during the course of medical treatment. It aims to enhance patient engagement, provide information, answer questions, offer reminders, and deliver personalized guidance related to a specific medical condition or treatment plan.

Medical treatment chatbots can be deployed in various healthcare settings, including hospitals, clinics, and even through patient-facing applications. They are typically designed to offer the following functionalities:

Education and Information: Treatment chatbots can provide patients with educational materials, resources, and information about their medical condition, medications, procedures, or lifestyle modifications. They can help patients understand their treatment plan better, its purpose, and expected outcomes.

Medication Reminders: Chatbots can assist patients in adhering to their medication schedules by sending reminders and notifications. They can help ensure that patients take their medications on time and in the prescribed dosage, thereby improving treatment effectiveness.

Symptom Monitoring: Patients can use chatbots to report and track their symptoms or side effects of medications. The chatbot may ask specific questions related to symptoms and provide recommendations based on the reported information. This data can also be shared with healthcare providers for monitoring and adjusting treatment plans.

Personalized Support: Treatment chatbots can offer personalized guidance and support based on an individual's medical history, treatment plan, and progress. They can provide suggestions for managing treatment-related challenges, offer lifestyle recommendations, and address common concerns or questions.

Appointment Scheduling: Chatbots can assist patients in scheduling appointments, arranging follow-up visits, or accessing telemedicine services. They can streamline the process and ensure that patients receive timely care.

Emotional Support: Some treatment chatbots are designed to provide emotional support and address the psychological aspects of medical treatment. They can offer encouragement, coping strategies, or connect patients to mental health resources if needed.

The specific capabilities of a medical treatment chatbot depend on its design, integration with healthcare systems, and the available data. It's important to note that while treatment chatbots can improve patient engagement and provide valuable support, they are not a substitute for direct medical advice or personalized care from healthcare professionals. They should be used as complementary tools to enhance the overall treatment experience.

According to a preferred embodiment, there is a mobile diagnostic support system for enhanced integration and involvement between a patient user and a care provider for provisioning medical care to a patient user comprising a plurality of user symptom reports (USR), one of said plurality of USR comprising a first user health condition report, a plurality of exam devices (ED), one of said plurality of ED comprising a vital sign monitor (VSM) having a first user exam result (UER), a plurality of lab tests (LT), one of said plurality of LT comprising a bodily fluid test (BFT) having a first user test result (UTR), a database comprising analysis data, said analysis data comprising a plurality of USR data, one of said plurality of USR data comprising a symptom report, a plurality of UER data, one of said plurality of UER data comprising a VSM exam result, a plurality of UTR data, one of said plurality of UTR data comprising a BFT test result, a plurality of diagnosis data (DD) one of said plurality of DD comprising a plurality of ED diagnosis interpretations (ED-DI) one of said plurality of ED-DI comprising a BPM diagnosis interpretation (BPM-DI) dataset, a second of said plurality of ED-DI comprising an LT diagnosis interpretation (LT-DI) dataset, a plurality of treatment data (TD), one of said plurality of TD comprising a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and a plurality of prophylaxis data (PD), one of said plurality of PD comprising a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset, an algorithm, a user device, a processor, and a care provider device, wherein optionally said user inputs at least one user health condition report, wherein said user connects at least one of said plurality of ED and/or said plurality of LT, wherein said processor uses said algorithm via encryption secured electronic transmission to connect to said plurality of ED and/or said plurality of LT, identify each of said plurality of ED and/or said plurality of LT, read a data from each of said plurality of ED and/or said plurality of LT, reformat/reorganize said data into a standard/consolidated formatted data-stream, and transmit said standard/consolidated data-stream into and/or attach to a patient record system, wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion, wherein said care provider device is used by a care provider to offer onsite diagnostic support, and wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

According to an alternate embodiment, there is a mobile diagnostic support system wherein said database further comprises a plurality of medical billing data (MBD), one of said plurality of MBD comprising a medical device billing data (MDBD), and wherein said processor uses said algorithm via encryption secured electronic transmission to display a billing suggestion.

According to an alternate embodiment, there is a mobile diagnostic support system further comprising a chatbot to review, assess, comment on, and/or adjust any of said plurality of USR, said plurality of UER, said plurality of UTR, said diagnosis suggestion, said treatment suggestion, and said prophylaxis suggestion.

According to an alternate embodiment, there is a mobile diagnostic support system further comprising a chatbot to review, assess, comment on, and/or adjust said plurality of MBD and/or said billing suggestion.

According to an alternate embodiment, there is a mobile diagnostic kit including the mobile diagnostic support system comprising: a backpack; a wheeled case; a stethoscope; a video camera; and a vital signs monitor comprising measurements for any combination of temperature, blood pressure, oxygen saturation, and/or heart rate.

According to an alternate embodiment, there is a mobile diagnostic kit further comprising any combination of: a blood chemistry analysis apparatus; and/or a hematology analysis apparatus.

According to an alternate embodiment, there is a mobile diagnostic kit further comprising: a urine analysis apparatus.

According to an alternate embodiment, there is a mobile diagnostic kit further comprising any combination of an electrocardiography analysis apparatus; a digital spirometer; and an ultrasound analysis apparatus.

According to an alternate embodiment, there is a mobile diagnostic kit further comprising any combination of: a blood clot analysis apparatus; an antigen presence analysis apparatus; a blood lipid analysis apparatus; a glycated hemoglobin analysis apparatus; and/or a rapid molecular test apparatus.

According to an alternate embodiment, there is a mobile diagnostic kit further comprising a recurring subscription fee.

According to a preferred embodiment, there is a method for enhanced integration and

involvement between a patient user and a care provider during a provisioning of medical care to a patient user comprising a plurality of user symptom reports (USR), one of said plurality of USR comprising a first user health condition report, a plurality of exam devices (ED), one of said plurality of ED comprising a vital sign monitor (VSM) having a first user exam result (UER), a plurality of lab tests (LT), one of said plurality of LT comprising a bodily fluid test (BFT) having a first user test result (UTR), a database comprising analysis data, said analysis data comprising a plurality of USR data, one of said plurality of USR data comprising a symptom report, a plurality of UER data, one of said plurality of UER data comprising a VSM exam result, a plurality of UTR data, one of said plurality of UTR data comprising a BFT test result, a plurality of diagnosis data (DD) one of said plurality of DD comprising a plurality of ED diagnosis interpretations (ED-DI) one of said plurality of ED-DI comprising a BPM diagnosis interpretation (BPM-DI) dataset, a second of said plurality of ED-DI comprising an LT diagnosis interpretation (LT-DI) dataset, a plurality of treatment data (TD), one of said plurality of TD comprising a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and a plurality of prophylaxis data (PD), one of said plurality of PD comprising a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset, an algorithm, a user device, a processor, and a care provider device, wherein optionally said user inputs at least one user health condition report, wherein said user connects at least one of said plurality of ED and/or said plurality of LT, wherein said processor uses said algorithm via encryption secured electronic transmission to connect to said plurality of ED and/or said plurality of LT, identify each of said plurality of ED and/or said plurality of LT, read a data from each of said plurality of ED and/or said plurality of LT, reformat/reorganize said data into a standard/consolidated formatted data-stream, and transmit said standard/consolidated data-stream into and/or attach to a patient record system, wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion, wherein said care provider device is used by a care provider to offer onsite diagnostic support, and wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

According to an alternate embodiment, there is a method wherein said database further comprises a plurality of medical billing data (MBD), one of said plurality of MBD comprising a medical device billing data (MDBD), and wherein said processor uses said algorithm via encryption secured electronic transmission to display a billing suggestion.

According to an alternate embodiment, there is a method further comprising a chatbot to review, assess, comment on, and/or adjust any of said plurality of USR, said plurality of UER, said plurality of UTR, said diagnosis suggestion, said treatment suggestion, and said prophylaxis suggestion.

According to an alternate embodiment, there is a method further comprising a chatbot to review, assess, comment on, and/or adjust said plurality of MBD and/or said billing suggestion.

According to a preferred embodiment, there is a mobile diagnostic kit for implementing a method for enhanced integration and involvement between a patient user and a care provider during a provisioning of medical care to a patient user comprising: a backpack; a wheeled case; a stethoscope; a video camera; and a vital signs monitor comprising measurements for any combination of temperature, blood pressure, oxygen satiation, and heart rate.

According to an alternate embodiment, there is a mobile diagnostic kit further comprising any combination of: a blood chemistry analysis apparatus; and/or a hematology analysis apparatus.

According to an alternate embodiment, there is a mobile diagnostic kit further comprising: a urine analysis apparatus.

According to an alternate embodiment, there is a mobile diagnostic kit further comprising any combination of an electrocardiography analysis apparatus; a digital spirometer; and an ultrasound analysis apparatus.

According to an alternate embodiment, there is a mobile diagnostic kit further comprising any combination of: a blood clot analysis apparatus; an antigen presence analysis apparatus; a blood lipid analysis apparatus; a glycated hemoglobin analysis apparatus; and/or a rapid molecular test apparatus.

According to an alternate embodiment, there is a mobile diagnostic kit further comprising a recurring subscription fee.

There are many advantages of the present invention. Most importantly, an advantage is that is it a unified POC remote patient care system that allows users an easy and convenient way to decrease diagnostic error due to the increased interaction between patient and care provider. The increase in interaction also leads to faster diagnosis, treatment, and prophylaxis decision support. The present invention offers due to the wide variety of imaging, testing, and results available and an onsite diagnostic support system. The present invention is a more complete POC remote patient care system that provides users with an easy and convenient way to decrease diagnostic error, seek and obtain faster diagnosis, treatment, and prophylaxis support decisions than are available today, and get onsite diagnostic support systems available to help both patients and care providers. Because of these advantages, the present invention makes it easier to reach rural environments and create decentralized clinical trials.

The interaction between a remote patient and a care provider takes place more interactively with the present invention. From the user's first interaction, the present invention offers enhanced connectivity between a remote patient and a care provider. Both participants are viewing and sharing the same information and both parties are able to leverage the full and complete record for use within the present interaction between a remote patient and a care provider.

The present invention offers much more integration and involvement between a remote patient and a care provider. There is no more of the usual serial fashion patient report and care provider questioning with the system of the present invention. This system offers the extensive tools for testing and results that immediately help ensure high speed accurate results leading to a unified POC remote patient care system that allows users an easy and convenient way to decrease diagnostic error, provide faster diagnosis, treatment, and prophylaxis support for decisions, and offer onsite diagnostic support system.

The invention has been described by way of examples only. Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the claims.

Although the invention has been explained in relation to various embodiments, it is to be understood that many other possible modifications and variations can be made without departing from the spirit and scope of the invention.

1. A mobile diagnostic support system for enhanced integration and involvement between a user and a care provider for provisioning medical care to said user comprising
a plurality of user symptom reports (USR), one of said plurality of USR comprising a first user health condition report,
a plurality of exam devices (ED), one of said plurality of ED comprising a vital sign monitor (VSM) having a first user exam result (UER),
a plurality of lab tests (LT), one of said plurality of LT comprising a bodily fluid test (BFT) having a first user test result (UTR),
a database comprising analysis data, said analysis data comprising
a plurality of USR data, one of said plurality of USR data comprising a symptom report,
a plurality of UER data, one of said plurality of UER data comprising a VSM exam result,
a plurality of UTR data, one of said plurality of UTR data comprising a BFT test result,
a plurality of diagnosis data (DD) one of said plurality of DD comprising
a plurality of ED diagnosis interpretations (ED-DI) one of said plurality of ED-DI comprising a BPM diagnosis interpretation (BPM-DI) dataset, a second of said plurality of ED-DI comprising an LT diagnosis interpretation (LT-DI) dataset,
a plurality of treatment data (TD), one of said plurality of TD comprising
a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and
a plurality of prophylaxis data (PD), one of said plurality of PD comprising
a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset,
an algorithm,
a user device,
a processor, and
a care provider device,
wherein optionally said user inputs at least one user health condition report,
wherein said user connects at least one of said plurality of ED and/or said plurality of LT,
wherein said processor uses said algorithm via encryption secured electronic transmission to
connect to said plurality of ED and/or said plurality of LT,
identify each of said plurality of ED and/or said plurality of LT,
read a data from each of said plurality of ED and/or said plurality of LT,
reformat/reorganize said data into a standard/consolidated formatted data-stream, and
transmit said standard/consolidated data-stream into and/or attach to a patient record system,
wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion,
wherein said care provider device is used by a care provider to offer onsite diagnostic support, and
wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

2. The mobile diagnostic support system of claim 1
wherein said database further comprises a plurality of medical billing data (MBD), one of said plurality of MBD comprising a medical device billing data (MDBD), and
wherein said processor uses said algorithm via encryption secured electronic transmission to display a billing suggestion.

3. The mobile diagnostic support system of claim 1 further comprising a chatbot to review, assess, comment on, and/or adjust any of said plurality of USR, said plurality of UER, said plurality of UTR, said diagnosis suggestion, said treatment suggestion, and said prophylaxis suggestion.

4. The mobile diagnostic support system of claim 2 further comprising a chatbot to review, assess, comment on, and/or adjust said plurality of MBD and/or said billing suggestion.

5. A mobile diagnostic kit including the mobile diagnostic support system of claim 1 comprising:
a backpack;
a wheeled case;
a stethoscope;
a video camera; and
a vital signs monitor comprising measurements for any combination of temperature, blood pressure, oxygen saturation, and/or heart rate.

6. The mobile diagnostic kit of claim 5 further comprising any combination of:
a blood chemistry analysis apparatus; and/or
a hematology analysis apparatus.

7. The mobile diagnostic kit of claim 5 further comprising:
a urine analysis apparatus.

8. The mobile diagnostic kit of claim 5 further comprising any combination of
an electrocardiography analysis apparatus;
a digital spirometer; and
an ultrasound analysis apparatus.

9. The mobile diagnostic kit of claim 5 further comprising any combination of:
a blood clot analysis apparatus;
an antigen presence analysis apparatus;
a blood lipid analysis apparatus;
a glycated hemoglobin analysis apparatus; and/or
a rapid molecular test apparatus.

10. The mobile diagnostic kit of claim 5 further comprising a recurring subscription fee.

11. A method for enhanced integration and involvement between a user and a care provider during a provisioning of medical care to said user comprising
a plurality of user symptom reports (USR), one of said plurality of USR comprising a first user health condition report,
a plurality of exam devices (ED), one of said plurality of ED comprising a vital sign monitor (VSM) having a first user exam result (UER),
a plurality of lab tests (LT), one of said plurality of LT comprising a bodily fluid test (BFT) having a first user test result (UTR),
a database comprising analysis data, said analysis data comprising
a plurality of USR data, one of said plurality of USR data comprising a symptom report,
a plurality of UER data, one of said plurality of UER data comprising a VSM exam result,
a plurality of UTR data, one of said plurality of UTR data comprising a BFT test result,
a plurality of diagnosis data (DD) one of said plurality of DD comprising
a plurality of ED diagnosis interpretations (ED-DI) one of said plurality of ED-DI comprising a BPM diagnosis interpretation (BPM-DI) dataset, a second of said plurality of ED-DI comprising an LT diagnosis interpretation (LT-DI) dataset,
a plurality of treatment data (TD), one of said plurality of TD comprising
a plurality ED treatment interpretations (ED-TI), one of said plurality of ED-TI comprising a BPM treatment interpretation (BPM-TI) dataset, a second of said plurality of ED-TI comprising an LT treatment interpretation (LT-TI) dataset, and
a plurality of prophylaxis data (PD), one of said plurality of PD comprising
a plurality of ED prophylaxis interpretations (ED-PI), one of said plurality of ED-PI comprising a BPM prophylaxis interpretation (BPM-PI) dataset, a second of said plurality of ED-PI comprising an LT prophylaxis interpretation (LT-PI) dataset,
an algorithm,
a user device,
a processor, and
a care provider device,
wherein optionally said user inputs at least one user health condition report,
wherein said user connects at least one of said plurality of ED and/or said plurality of LT,
wherein said processor uses said algorithm via encryption secured electronic transmission to
connect to said plurality of ED and/or said plurality of LT,
identify each of said plurality of ED and/or said plurality of LT,
read a data from each of said plurality of ED and/or said plurality of LT,
reformat/reorganize said data into a standard/consolidated formatted data-stream, and
transmit said standard/consolidated data-stream into and/or attach to a patient record system,
wherein said processor uses said algorithm to compare said plurality of USR, said plurality of UER, and said plurality of UTR to said plurality of DD, said plurality of TD, and said plurality of PD and displays on said care provider device and/or said user device at least one of a diagnosis suggestion, a treatment suggestion, or a prophylaxis suggestion,
wherein said care provider device is used by a care provider to offer onsite diagnostic support, and
wherein said care provider device is used by a care provider to review, assess, comment on, and/or adjust any of said plurality of USR, said plurality of UER, said plurality of UTR, said diagnosis suggestion, said treatment suggestion, and said prophylaxis suggestion.

12. The method of claim 11
wherein said database further comprises a plurality of medical billing data (MBD), one of said plurality of MBD comprising a medical device billing data (MDBD), and
wherein said processor uses said algorithm via encryption secured electronic transmission to display a billing suggestion.

13. The method of claim 11 further comprising a chatbot to review, assess, comment on, and/or adjust any of said plurality of USR, said plurality of UER, said plurality of UTR, said diagnosis suggestion, said treatment suggestion, and said prophylaxis suggestion.

14. The method of claim 12 further comprising a chatbot to review, assess, comment on, and/or adjust said plurality of MBD and/or said billing suggestion.

15. A mobile diagnostic kit for implementing a method for enhanced integration and involvement between a user and a care provider during a provisioning of medical care to said user comprising:
a backpack;
a wheeled case;
a stethoscope;
a video camera; and
a vital signs monitor comprising measurements for any combination of temperature, blood pressure, oxygen satiation, and heart rate.

16. The mobile diagnostic kit of claim 15 further comprising any combination of:
a blood chemistry analysis apparatus; and/or
a hematology analysis apparatus.

17. The mobile diagnostic kit of claim 15 further comprising:
a urine analysis apparatus.

18. The mobile diagnostic kit of claim 15 further comprising any combination of
an electrocardiography analysis apparatus;
a digital spirometer; and
an ultrasound analysis apparatus.

19. The mobile diagnostic kit of claim 15 further comprising any combination of:
a blood clot analysis apparatus;
an antigen presence analysis apparatus;
a blood lipid analysis apparatus;
a glycated hemoglobin analysis apparatus; and/or
a rapid molecular test apparatus.

20. The mobile diagnostic kit of claim 15 further comprising a recurring subscription fec.
